(19) United States
(12) Patent Application Publication
(10) Pub. No.: US 2024/0108557 A1
(43) Pub. Date: Apr. 4, 2024

(54) TOPICAL COMPOSITION USEFUL FOR THE DAILY CARE OF ACNE-PRONE SKIN

(71) Applicant: Pierre Fabre Dermo-Cosmetique, Boulogne-Billancourt (FR)

(72) Inventors: Fernanda CHAVES, Gavea Rio De Janeiro (BR); Juliana AMADO, Catete Rio De Janeiro (BR); Anne Laure GAUDRY, Balma (FR); Hélène DUPLAN, Auzeville Tolosan (FR); Martine MAITRE, Tournefeuille (FR)

(73) Assignee: Pierre Fabre Dermo-Cosmetique, Boulogne-Billancourt (FR)

(21) Appl. No.: 18/274,595

(22) PCT Filed: Jan. 28, 2022
(86) PCT No.: PCT/EP2022/052102

§ 371 Date: Jul. 27, 2023

(30) Foreign Application Priority Data
Jan. 29, 2021 (EP) 21305125.3

(51) Int. Cl. A61K 8/362; A61K 8/365; A61K 8/37; A61K 8/9789; A61K 8/9794; A61Q 19/00

(52) U.S. Cl. CPC A61K 8/362; A61K 8/365; A61K 8/375; A61K 8/9789; A61K 8/9794; A61Q 19/00; A61K 2800/30; A61K 2800/592

(57) ABSTRACT

The present disclosure relates topical compositions, such as cosmetic topical compositions, comprising a keratolytic combination of succinic acid and lactic acid and their use for the daily care of oily skins, acne-prone skins, acne-affected skins and acne-treated skins.

### FIELD OF THE INVENTION

The invention relates to topical compositions, such as cosmetic topical compositions, comprising a keratolytic combination of succinic acid and lactic acid and their use for the daily care of oily skins, acne-prone skins, acne-affected skins and acne-treated skins.

### BACKGROUND OF THE INVENTION

Acne is the most common chronic disease of the pilosebaceous follicle of human skin, caused by multiple factors and leading to the appearance of various types of lesions. It is a chronic immunoinflammatory disease, affecting about 900 million people worldwide, mostly adolescents (prevalence of 80% to 85%), of both sexes, but also many adult women. With multifactorial etiological cause, acne is a dermatological condition that causes physical and emotional changes in the affected individuals as a result of the unsightly appearance that the skin presents due to the formation of comedones, papules, cyst, nodules and pustules that tend to generate hypertrophic scars in the skin. (LIMA, 2006; MANFRINATO, 2009). This multifactorial etiology with genetic influence involves four phenomena: inflammatory process in all its phases, dyskeratosis of the distal portion of the follicular orifice, diseborrhea (quantitative and qualitative alteration of sebum), androgenic hormonal influence and presence of Cutibacterium acnes bacteria, with virulence dependent on the follicular environment and high antigenic power for receptors present in the cell membrane that are part of the patient's innate immunity.

Depending on the degree of affection or clinical evolution, the different types of acne can be classified into non-inflammatory or inflammatory acne, of mild, moderate or severe degrees (MANFRINATO, 2009). Non-inflammatory acne is characterized by the presence of comedones, without an inflammatory condition, whereas inflammatory acne is classified in four degrees, according to the intensity, quantity and characteristics of the lesions:
Grade I acne (Comedogenic acne—non-inflammatory): oily skin, open and closed comedones without inflammatory lesions;
Grade II acne (inflammatory papulopustular acne): open and closed comedones, papules and pustules;
Grade III acne (inflammatory nodular-cystic acne): oily skin, open and closed comedones, pustules, nodules and cysts;
Grade IV acne (Acne fulminans): Infectious and systemic form of acne, of unknown cause and abrupt onset, which predominantly affects males. Although rare, it is devastating and serious. The etiology of fulminant acne is not the same as that of acne vulgaris. It does not occur with the same process of obstruction of the pilosebaceous follicle, hypersecretion and hormonal factors. In some cases, the affected individual has papules, pustules and nodules that evolve into ulcers.

Daily skin care, including daily skin cleansing, is considered to be an important component in the successful management of acne. Medical treatments may cause in the long-term adverse effects, such as erythema, peeling, edema, dryness, harshness, or cause symptoms, such as itching and burning. These reactions can result from direct effects caused by the active ingredient (e.g., retinoids, benzoyl peroxide, some topical antibiotics, etc) or by the characteristics of the vehicle. The choice of proper daily skin care products is therefore of uttermost importance to avoid further insulting an already sensitized skin. In addition, a proper choice of skin care products may increase the compliance of individuals to the dermatological treatment and refrain individuals from over cleansing the skin which may stimulate production of sebum and ultimately increase the severity of acne.

Though many skin care products are available on the market, a need remains for a topical composition that is gentle to the skin and that may help improving the condition of oily skin, acne-prone skin, acne-affected skin and/or acne-treated skin.

### SUMMARY OF THE INVENTION

The invention relates to a topical composition comprising:
a. a combination of keratolytic agents including succinic acid and one or more alpha hydroxy acids, preferably wherein the alpha hydroxy acids consist of lactic acid;
b. one or more physiologically acceptable carriers and/or adjuvants;
   wherein the total amount of succinic acid and alpha hydroxy acids ranges from about 1 to about 8% by weight relative to the total weight of the composition and wherein the succinic acid/total alpha hydroxy acids mass ratio is superior or equal to 1.

The invention also relates to the use of a topical composition as disclosed herein for cleansing oily skin, acne-prone skin, acne-affected skin and/or acne-treated skin, for controlling oiliness of skin and/or for reducing Stratum corneum stiffness while controlling epidermis integrity.

The invention also relates to a cosmetic method for cleansing the skin, said method comprising:
a. applying a composition as disclosed herein to previously moistened skin,
b. optionally emulsifying with water,
c. rinsing the skin, and
d. drying the skin.

Further aspects of the invention are as disclosed herein and in the claims.

### FIGURES

FIG. 1A: Measures and analysis of the elasticity of the Stratum corneum. Percentage of inhibition of elastic modulus compared to control (mean of two donors): control—untreated-, salicylic acid 1%, succinic acid 0.6%, lactic acid 0.36%, and lactic acid 0.36%+succinic acid 0.6% (% by weight).

FIG. 1B: Measures and analyse of cell-cell cohesion of epidermis deep layer (control of epidermis integrity). Percentage of inhibition of elastic modulus compared to salicylic acid (mean of two donors): salicylic acid 1%, lactic acid 0.36%+succinic acid 0.6% (% by weight).

FIG. 2: Global lesion count on trunk, Error bars: 95% CI, ***p-values≤0.001.

### DEFINITIONS

The term “about” means in the context of the present invention that the concerned value may be lower or higher by 10%, especially by 5%, in particular by 1%, than the indicated value. It encompasses the indicated value and values that may be lower or higher by 10%, especially by 5%, in particular by 1%, than the indicated value. As a matter of examples, when a range is said to vary from about X to about Y, it includes the range from X to Y and optionally values that may be lower by 10%, especially by 5%, in particular by 1%, than X and values that may be higher by 10%, especially by 5%, in particular by 1% than Y. The term “keratolytic agent” as used herein refers to an agent that softens, disrupts, dissolves, solubilizes, or loosens a keratinized obstruction.

### DESCRIPTION OF THE INVENTION

Surprisingly, it has been found that topical compositions comprising a combination of succinic acid and one or more alpha hydroxy acids, in particular lactic acid as disclosed herein, efficiently improve the condition of acne-prone skin, acne-affected skin and/or acne-treated skin thanks to the combined bactericidal and keratolytic action of succinic acid and alpha hydroxy acid(s), in particular lactic acid, while preserving epidermis integrity.

“Alpha hydroxy acid” as used herein designates organic carboxylic acids in which one hydroxyl group is attached to the carbon in alpha of the carboxylic acid function. Examples of suitable alpha hydroxy acids include, but are not limited to, lactic acid, glycolic acid, tartaric acid, malic acid, citric acid and mandelic acid.

In some aspects, the topical compositions of the invention comprise:
a. a combination of keratolytic agents including succinic acid and one or more alpha hydroxy acids, preferably selected from the group consisting of lactic acid, glycolic acid, tartaric acid, malic acid, citric acid, mandelic acid and combinations thereof;
b. one or more physiologically acceptable carriers and/or adjuvants;
   wherein the total amount of succinic acid and total alpha hydroxy acids ranges from about 1 to about 8% by weight relative to the total weight of the composition and wherein the succinic acid/total alpha hydroxy acids mass ratio is superior or equal to 1, preferably superior or equal to 1.5.

In some preferred aspects, the topical compositions of the invention comprise:
a. a combination of keratolytic agents including succinic acid and alpha hydroxy acids, wherein the alpha hydroxy acids consist of lactic acid;
b. one or more physiologically acceptable carriers and/or adjuvants;
   wherein the total amount of succinic acid and lactic acid ranges from about 1 to about 8% by weight relative to the total weight of the composition and wherein the succinic acid/lactic acid mass ratio is superior or equal to 1, preferably superior or equal to 1.5.

By “topical composition” as used herein, it is meant a composition for application to the skin of humans. The topical composition may be a cosmetic topical composition or a pharmaceutical/dermatological topical composition.

By “cosmetic topical compositions” as used herein, it is meant a skin care composition. Skin care compositions are generally used to cleanse, protect, moisturize the skin and/or to treat the skin, i.e., to administer beneficial agents in order to improve the condition of the skin to which they are applied.

The topical compositions of the invention may be in any suitable form for skin care. For instance, the topical composition may be in the form of a solution, micellar solution, lotion, emulsion, suspensions, cream, ointment, serum, mask, foam or gel. Solution, lotion, foam and gel may be preferred. The choice of suitable carriers and adjuvants as described herein will largely depend on the selected form of the composition.

The topical composition preferably has a pH that ranges from 4.5 to 5.1, e.g., from 4.8 to 5.1.

The compositions of the invention are particularly suitable for cleansing oily skin or the skin of acne-prone, acne-affected and/or acne-treated individuals. Thus, in some preferred embodiments, the invention relates to a topical cleansing composition comprising:
a. a combination of keratolytic agents including succinic acid and one or more alpha hydroxy acids, preferably selected from the group consisting of lactic acid, glycolic acid, tartaric acid, malic acid, citric acid, mandelic acid and combinations thereof;
b. one or more physiologically acceptable carriers and/or adjuvants;
   wherein the total amount of succinic acid and alpha hydroxy acids ranges from about 1 to about 8% by weight relative to the total weight of the composition; and wherein the succinic acid/total alpha hydroxy acids mass ratio is superior or equal to 1, preferably superior or equal to 1.5.

In some preferred embodiments, the invention relates to a topical cleansing composition comprising:
a. a combination of keratolytic agents including succinic acid and alpha hydroxy acids, wherein the alpha hydroxy acids consist of lactic acid;
b. one or more physiologically acceptable carriers and/or adjuvants;
   wherein the total amount of succinic acid and lactic acid ranges from about 1 to about 8% by weight relative to the total weight of the composition; and wherein the succinic acid/lactic acid mass ratio is superior or equal to 1, preferably superior or equal to 1.5.

Topical cleansing compositions comprising a combination of succinic acid and alpha hydroxy acids, in particular lactic acid, as disclosed herein, were found to efficiently and gently cleanse the skin without causing local skin reactions (e.g. drying, irritations) and without resulting in over-compensation of sebum production. The topical cleansing compositions efficiently remove dirt, pollution and sebum excess. Oiliness and shininess of the skin are instantly reduced with observed long lasting mattifying effects and/or anti-blemish effects. In addition, as previously mentioned and as further detailed herein below, the topical cleansing compositions help improving the condition of oily skin, acne-prone skin, acne-affected skin and acne-treated skin.

The topical cleansing composition is preferably an aqueous-based composition, i.e., the topical cleansing composition is not an oil-based composition, nor an emulsion-type composition nor a solvent-based composition (e.g., alcohol-based composition). Preferred topical cleansing compositions are monophase compositions.

The topical cleansing composition is preferably a liquid. Preferably, the topical cleansing composition may have a viscosity at ambient temperature (25° C.) that varies within a broad range, for example a viscosity ranging from 500 to 3500 cPs, preferably from 1400 to 3500 cPs. The viscosity is generally measured at 25° C., using a viscosimeter Brookfield DV1RV.

In some embodiments, the topical cleansing composition is in the form of an aqueous gel.

The topical cleansing composition is generally a rinse-off composition. By “rinse-off composition”, it is meant a composition that is applied to the skin for a short period of time (few seconds or minutes) and then rinsed off with water.

The topical compositions may be supplied as a liquid, which may be dispensed from a package onto an implement (e.g., cotton pads, cloths) or directly onto the skin. Alternatively, the topical composition may be absorbed onto wipes.

Components of the topical compositions, in particular of the topical cleansing compositions, are as described herein below.

Keratolytic Agents

The topical compositions comprise a combination of keratolytic agents which includes succinic acid and one or more alpha hydroxy acids, in particular lactic acid. It was found that succinic acid and one or more alpha hydroxy acids, in particular lactic acid, delivers a synergistic effect allowing to efficiently soften and separate the cornified epithelium of the skin (cohesiveness of Stratum corneum is relaxed) causing desquamation of the skin while preserving epidermis integrity. Resultantly, reduced stiffness of the skin may be observed. The keratolytic agents help removing comedone plugs (i.e., solid, horny masses of packed keratinized cells that clog follicles) and reduce continued accumulation of keratinized cells that contribute to enlargement of comedones. Resultantly, a reduced pore size may be observed.

Furthermore, the combination of succinic acid and one or more alpha hydroxy acids, in particular lactic acid, delivers a bactericidal action. Succinic acid and one or more alpha hydroxy acids, in particular lactic acid, were shown to be effective in targeting Cutibacterium acnes, Staphylococcus aureus and Staphylococcus epidermis that may have a causal role in acne, although Cutibacterium acnes is the main trigger of acne.

Typically, the topical composition is free from further keratolytic agents.

In some embodiments, the topical composition is free from salicylic acid.

In some embodiments, the topical composition is free from salicylic acid derivatives.

In some embodiments, the topical composition is free from resorcinol, sulfur and benzoyl peroxide.

In preferred embodiments, the alpha hydroxy acids consist of lactic acid.

In preferred embodiments, the combination of keratolytic agents consists of succinic acid and lactic acid.

The total amount of succinic acid and alpha hydroxy acids in the topical composition ranges from about 1 to 8% by weight, e.g. from about 2 to 8% by weight, or from about 3 to 8% by weight, or from about 4 to 8% by weight, or from about 4 to 7% by weight, or from about 4 to 6% by weight, or is about 5%, by weight relative to the total weight of the composition.

When the alpha hydroxy acids consist of lactic acid, the total amount of succinic acid and lactic acid in the topical composition ranges from about 1 to 8% by weight, e.g. from about 2 to 8% by weight, or from about 3 to 8% by weight, or from about 4 to 8% by weight, or from about 4 to 7% by weight, or from about 4 to 6% by weight, or is about 5%, by weight relative to the total weight of the composition.

When the topical compositions do not comprise further keratolytic agents, the topical compositions comprise from about 1 to 8% by weight, e.g. from about 2 to 8% by weight, or from about 3 to 8% by weight, or from about 4 to 8% by weight, or from about 4 to 7% by weight, or from about 4 to 6% by weight, or about 5% by weight of a combination of keratolytic agents relative to the total weight of the composition.

The succinic acid/total alpha hydroxy acids mass ratio is superior or equal to 1, preferably superior or equal to 1.5, more preferably superior or equal to 1.6. Preferably, the succinic acid/total alpha hydroxy acids mass ratio is inferior or equal to 4, or inferior or equal to 3, or inferior or equal to 2 or inferior or equal to 1.7.

When the alpha hydroxy acids consist of lactic acid, the succinic acid/lactic acid mass ratio is superior or equal to 1, preferably superior or equal to 1.5, more preferably superior or equal to 1.6. Preferably, the succinic acid/lactic acid mass ratio is inferior or equal to 4, or inferior or equal to 3, or inferior or equal to 2 or inferior or equal to 1.7.

In some embodiments, the topical compositions comprise from about 0.5% to about 6%, preferably from about 1% to about 6%, or from about 2% to about 6%, or from about 3 to about 6%, or from about 2% to about 4%, by weight of succinic acid and from about 0.5% to about 5%, preferably from about 1% to about 5%, or from about 1% to about 4% or from about 2 to about 5% or from about 1% to about 3%, by weight of alpha hydroxy acids relative to the total weight of the composition.

In some embodiments, when the alpha hydroxy acids consist of lactic acid, the topical compositions comprise from about 0.5% to about 6%, preferably from about 1% to about 6%, or from about 2% to about 6%, or from about 3 to about 6%, or from about 2% to about 4%, by weight of succinic acid and from about 0.5% to about 5%, preferably from about 1% to about 5%, or from about 1% to about 4% or from about 2 to about 5% or from about 1% to about 3%, by weight of lactic acid relative to the total weight of the composition.

Physiologically Acceptable Carriers and/or Adjuvants

The compositions of the invention being intended for topical application, the one or more carriers and/or excipients should be physiologically acceptable.

The term “physiologically acceptable” means compatible with the skin (e.g., body, face, eyelids) and mucous membranes (e.g., lips), i.e., it does not induce undue toxicity, incompatibility, instability, irritation, allergic response, or the like.

The choice of suitable carriers and adjuvants will largely depend on the selected form of the topical composition.

Carriers

The topical compositions may comprise from about 10% to about 90%, e.g., from about 10% to about 70% or from about 15% to about 60% by weight of the composition of one or more carriers.

Though the physiology acceptable carrier could be an organic solvent (e.g., propylene glycol, polyethylene glycol, polypropylene glycol, glycerol, 1,2,4-butanetriol, sorbitol esters, 1,2,6-hexanetriol, ethanol, and mixtures thereof), a silicone solvent, oils, lipids and/or waxes, the physiologically acceptable carrier of the topical compositions is typically water. Preferably, water is used as the sole carrier. The topical composition is then preferably an aqueous-based composition, i.e., the topical composition is not an oil-based composition nor an emulsion-type composition nor a solvent-based composition (e.g., alcohol-based composition). Hence, in preferred embodiments, the topical composition is free from oily phase and from oil-containing components.

Adjuvants

The topical compositions may also comprise adjuvants that are common in the cosmetic or dermatological field, such as surfactants, thickeners, gelling agents, moisturizers, active agents (e.g., plants extracts, vitamins), preservatives, emulsifiers, fragrances. The amounts of these various adjuvants are those conventionally used in the field under consideration, for example from about 0.0001% to about 30%, or from about 0.0001% to about 20%, by weight relative to the total weight of the topical composition, or from about 0.01% to about 20% relative to the total weight of the topical composition.

The topical compositions being preferably free from oily phase and oil-containing components, the topical composition is typically free from emulsifiers. Emulsifiers may contribute the dehydration of the skin.

The topical compositions may comprise one or more surfactants. The surfactant may be a polymeric surfactant or a non-polymeric one. Surfactants provide cleaning benefits, lather properties and rheology properties to the topical compositions. The surfactant or combinations of surfactants is typically mild, which means that the surfactants provide sufficient cleaning but do not overly dry the skin.

The total surfactants amount in the topical composition may range from about 1% to about 30%, e.g., from about 1% to 20% or from about 1% to 15%, by weight relative to the total weight of the composition. The surfactants may be cationic, anionic, zwitterionic, amphoteric, nonionic, or a combination thereof. Though in principle any surfactant class may be used, the topical compositions when used in cleansing application will typically be formulated with anionic surfactants or with a combination of one or more anionic surfactants with one or more surfactants selected from the other surfactant classes. Thus, the topical composition, in particular the topical cleansing composition, may comprise from about 1% to about 20%, e.g., from about 1% to about 18% or from about 1% to about 15% by weight of one or moreanionic surfactants and from about 1% to about 15%, e.g., from about 1% to about 12% or from about 1% to about 10% by weight of surfactant(s) selected from the other surfactant classes, for instance a non ionic surfactant.

A wide variety of anionic surfactants are useful herein. The anionic surfactant may contain any counterion such as sodium, potassium, ammonium, triethanolamine, etc. Non-limiting examples of anionic surfactants include those selected from the group consisting of carboxylates, sarcosinates, sulfates, sulfonates, isethionates, taurates, phosphates, lactylates, citrates, glutamates, and mixtures thereof. Preferences may be given to anionic surfactants selected from the group consisting of carboxylates, sarcosinates, isethionates, taurates, phosphates, lactylates, citrates, glutamates, and mixtures thereof or to anionic surfactants selected from the group consisting of carboxylates, taurates, lactylates, citrates, glutamates or to anionic surfactants selected from the group consisting of lactylates, citrates, glutamates, and mixtures thereof, in particular glutamate. Suitable anionic surfactants include but are not limited to amino acid-based anionic surfactant, such disodium cocoyl glutamate. Preferably, the surfactant is disodium cocoyl glutamate, disodium coco glucoside citrate or combinations thereof.

Nonionic surfactants useful herein include, but are not limited to, those selected from the group consisting of alkyl glucosides, alkyl polyglucosides, polyhydroxy fatty acid amides, alkoxylated fatty acid esters, alkoxylated fatty alcohol ethers, sucrose esters, and mixtures thereof. Preferred nonionic surfactant is decyl glucoside.

Amphoteric surfactants useful herein include, but are not limited to, those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Useful amphoteric surfactants include the group consisting of cocoamphoacetate, cocoamphodiacetate, lauroamphoacetate, lauroamphodiacetate, and mixtures thereof. In some embodiments, the topical composition is free from amphoteric surfactant.

Zwitterionic surfactants useful herein include, but are not limited to, those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Useful zwitterionic surfactants include betaines, amphoacetates and sulfobetaines, e.g., cocoamidopropylbetaine, sodium laurylamphoacetate and cocoamidopropylhydroxysultaine. In some embodiments, the topical composition is free from zwitterionic surfactant.

In some embodiments, the topical compositions comprise as surfactants at least one amino acid-based surfactant, preferably at least disodium cocoyl glutamate. The amount of the amino acid-based surfactant may range from about 0.1% to about 12% or from about 2 to about 10%, preferably from about 5% to about 10% by weight relative to the total weight of the composition.

In some embodiments, the topical compositions comprise at least one amino acid-based surfactant, preferably at least disodium cocoyl glutamate, in proportions that may be as disclosed herein and at least one surfactant which is different from an amino acid-based surfactant, for instance at least disodium coco glucoside citrate or at least decyl glucoside. Preferably, the topical compositions comprise a mixture of anionic and non ionic surfactants. It should then be understood that the topical compositions is free from amphoteric surfactant and zwitterionic surfactant. More specifically, in some embodiments, the topical composition comprises a mixture of disodium cocoyl glutamate, disodium coco glucoside citrate and decyl glucoside.

Typically, the mixture comprises from about 0.1% to about 12% of disodium cocoyl glutamate, from about 0.5% to about 6% of disodium coco glucoside citrate and from about 1% to about 15% of decyl glucoside relative to the total weight of the composition.

In some embodiments, the mass ratio between the one or more anionic surfactants and other classes of surfactants, e.g. non ionic surfactant, is about 50/50.

The topical composition may comprise thickeners, such as cellulose derivatives (e.g., hydroxymethyl cellulose and hydroxypropyl cellulose), starches and starch derivatives, acrylic acid and acrylate polymers and copolymers, polyethylene glycol derivatives, natural gums (e.g., xanthan gum, scleroglucan gum and/or carrageenan gum) and salts (e.g., sodium chloride). In some embodiments, the thickener is Ceteareth 60 myristyl glycol.

The topical composition may comprise preservatives, such as benzoic acid or salt thereof, benzyl alcohol, sorbic acid or salt thereof, ureas (e.g., imidazolidinyl urea, diazolidinyl urea), parabens, dehydroacetic acid, sodium dehydroacetate, PHMB (polyhexamethylene biguanide), phenoxyethanol, ethylhexylglycerol, salicylic acid or salt thereof and sodium benzoate.

The topical composition may comprise further active agents. Active agents are agents that further improve the condition of the skin to which they are applied.

Suitable active agents may be anti-seborrheic agents and/or pore refiners. Examples of anti-seborrheic agents include but are not limited to 2,3-dihydroxypropyl dodecanoate, sabal extract, pumpkin seed oil, extract of urtica dioic and combinations thereof, preferably 2,3-dihydroxypropyl dodecanoate. Examples of pore refiners include but are not limited to Lens esculenta seed extract.

However, the topical composition is typically free from anti-acneic agents, in particular free from ichtyol, retinoic acid and/or zinc gluconate.

In some embodiments, the topical composition comprises the following components:

Composition
(% by weight relative to the weight of the composition)

| | | |
|---|---|---|
| Succinic acid | Keratolytic agent | 0.5-6% |
| Lactic acid | Keratolytic agent | 0.5-5% |
| Disodium cocoyl glutamate | Anionic surfactant | 0.1-12% |
| Disodium coco glucoside | Anionic surfactant | 0.5-6% |
| citrate | | |
| Decyl glucoside | Non ionic surfactant | 1.0-15% |
| Glyceryl laurate | Antiseborrheic agent | 0.1-1% |
| Lens esculenta seed extract | Pore refiner agent | 0.005-0.1% |
| Ceteareth 60 myristyl glycol | Thickening agent | 0.5-4% |
| Sodium hydroxyde | | Qsp pH |
| | | 4.8-5.2 |
| Water | | Qsp 100 |

### FURTHER EMBODIMENTS OF THE INVENTION

In some further aspects, the invention relates to topical compositions comprising:
a. a combination of keratolytic agents including succinic acid and one or more alpha hydroxy acids;
b. one or more physiologically acceptable carriers and/or adjuvants;
   wherein the total amount of succinic acid and alpha hydroxy acids ranges from about 1 to about 8% by weight relative to the total weight of the composition.

In some embodiments, the alpha hydroxy acids are selected within the group consisting of lactic acid, glycolic acid, tartaric acid, malic acid, citric acid, mandelic acid and combinations thereof, more preferably the alpha hydroxy acid is lactic acid.

The total amount of succinic acid and alpha hydroxy acids in the topical composition may range from about 2 to 8% by weight, or from about 3 to 8% by weight, or from about 4 to 8% by weight, or from about 4 to 7% by weight, or from about 4 to 6% by weight, or is about 5%, by weight relative to the total weight of the composition.

In some preferred embodiments, the succinic acid/total alpha hydroxy acids mass ratio of the topical compositions is superior or equal to 1, preferably superior or equal to 1.5, more preferably superior or equal to 1.6. Preferably, the succinic acid/total alpha hydroxy acids mass ratio is inferior or equal to 4, or inferior or equal to 3, or inferior or equal to 2 or inferior or equal to 1.7.

In some embodiments, the topical compositions comprise from about 0.5% to about 6%, preferably from about 1% to about 6%, or from about 2% to about 6%, or from about 3 to about 6%, or from about 2% to about 4%, by weight of succinic acid and from about 0.5% to about 5%, preferably from about 1% to about 5%, or from about 1% to about 4% or from about 2 to about 5% or from about 1% to about 3%, by weight of total alpha hydroxy acids relative to the total weight of the composition.

In some preferred embodiments, the alpha hydroxy acids consist of lactic acid.

In some further aspects, the invention relates to a topical cleansing composition comprising:
a. a combination of keratolytic agents including succinic acid and one or more alpha hydroxy acids;
b. one or more physiologically acceptable carriers and/or adjuvants;
   wherein the total amount of succinic acid and alpha hydroxy acids ranges from about 1 to about 8% by weight relative to the total weight of the composition.

In some embodiments, the alpha hydroxy acids are selected within the group consisting of lactic acid, glycolic acid, tartaric acid, malic acid, citric acid, mandelic acid and combinations thereof, more preferably the alpha hydroxy acid is lactic acid.

The total amount of succinic acid and alpha hydroxy acids in the topical cleansing compositions may range from about 2 to 8% by weight, or from about 3 to 8% by weight, or from about 4 to 8% by weight, or from about 4 to 7% by weight, or from about 4 to 6% by weight, or is about 5%, by weight relative to the total weight of the composition.

In some preferred embodiments, the succinic acid/total alpha hydroxy acids mass ratio of the topical cleansing compositions is superior or equal to 1, preferably superior or equal to 1.5, more preferably superior or equal to 1.6. Preferably, the succinic acid/total alpha hydroxy acids mass ratio is inferior or equal to 4, or inferior or equal to 3, or inferior or equal to 2 or inferior or equal to 1.7.

In some embodiments, the topical cleansing compositions comprise from about 0.5% to about 6%, preferably from about 1% to about 6%, or from about 2% to about 6%, or from about 3 to about 6%, or from about 2% to about 4%, by weight of succinic acid and from about 0.5% to about 5%, preferably from about 1% to about 5%, or from about 1% to about 4% or from about 2 to about 5% or from about 1% to about 3%, by weight of alpha hydroxy acids relative to the total weight of the composition.

In some preferred embodiments, the alpha hydroxy acids consist of lactic acid.

Further aspects of these topical compositions may be as disclosed herein above, e.g., in the sections “Keratolytic agents” and “Physiologically acceptable carriers and/or adjuvants”.

Methods and Uses

The topical composition may be used for protecting, cleansing, moisturizing, treating oily skin, acne-prone skin, acne-affected skin and/or acne-treated skin. More specifically, the topical cleansing composition may be used for cleansing oily skin, acne-prone skin, acne-affected skin and/or acne-treated skin.

Hence, the invention relates to the cosmetic use of a topical composition as disclosed herein for protecting, cleansing or moisturizing oily skin, acne-prone skin, acne-affected skin and/or acne-treated skin.

The present invention also relates to the cosmetic use of a topical composition as disclosed herein for controlling the oiliness of skin or for delivering a prolonged antibacterial action or for beneficially acting on the cutaneous flora or for decreasing the Stratum corneum stiffness while controlling the epidermis integrity.

The cosmetic use comprises applying an effective amount of a topical composition as described herein to an individual in need thereof, in particular to the skin of the face or of the trunk. The application may be performed daily, for example at a rate of a two applications per day, for example once in the morning and once in the evening. The application may be performed over a time period ranging from one week to several weeks, or even several months, preferably for at least three weeks.

The invention also relates to a cosmetic (non-therapeutic) method for improving oily skin, acne-prone skin, acne-affected skin and/or acne-treated skin conditions which comprises the application of an effective amount of a topical composition as described herein to an individual in need thereof.

The invention also relates to a cosmetic method for cleansing the skin, said method comprising
a. applying a composition as disclosed herein to the previously moistened skin, preferably twice a day (e.g., morning and evening);
b. optionally emulsifying with water;
c. rinsing, typically thoroughly; and
d. drying the skin.

The application may be repeated for at least three weeks, preferably for at least four weeks, to obtain a long-lasting effect.

The present invention also relates to the therapeutic use of the topical composition as disclosed herein.

The invention also relates to the use of a topical composition as disclosed herein in the treatment of acne.

The invention also relates to the use of a topical composition as disclosed herein in the preparation of a medicament for the treatment of acne.

The invention also relates to a method for treating acne which comprises the application of an effective amount of a topical composition as described herein to an individual in need thereof. The invention also relates to a topical composition as disclosed herein for use in the treatment of acne.

Embodiments of the present invention will now be described by way of the following examples which are provided for illustrative purposes only, and not intended to limit the scope of the disclosure.

### EXAMPLES

### Example 1A

Composition
% by weight of the composition

| | | |
|---|---|---|
| Succinic acid | Keratolytic agent | 3-5% |
| Lactic acid | Keratolytic agent | 1.5-3.5% |
| Disodium cocoyl glutamate | Anionic surfactant | 0.1-12% |
| Disodium coco glucoside | Anionic surfactant | 0.5-6% |
| citrate | | |
| Decyl glucoside | Non ionic surfactant | 1.0-15% |
| Glyceryl laurate | Antiseborrheic agent | 0.1-1% |
| Lens esculenta seed extract | Pore refiner agent | 0.005-0.1% |
| Ceteareth 60 myristyl glycol | Thickening agent | 0.5-4% |
| Sodium hydroxyde | | Qsp pH |
| | | 4.8-5.2 |
| Water | | Qsp 100 |

### Example 1B

Composition
% by weight of the composition

| | | |
|---|---|---|
| Succinic acid | Keratolytic agent | 3% |
| Lactic acid | Keratolytic agent | 1.8% |
| Disodium cocoyl glutamate | Anionic surfactant | 0.1-12% |
| Disodium coco glucoside | Anionic surfactant | 0.5-6% |
| citrate | | |
| Decyl glucoside | Non ionic surfactant | 1.0-15% |
| Glyceryl laurate | Antiseborrheic agent | 0.1-1% |
| Lens esculenta seed extract | Pore refiner agent | 0.005-0.1% |
| Ceteareth 60 myristyl glycol | Thickening agent | 0.5-4% |
| Sodium hydroxyde | | Qsp pH |
| | | 4.8-5.2 |
| Water | | Qsp 100 |

### Example 2: Synergistic Keratolytic Effect of the Combination Succinic Acid and Lactic Acid Wherein the Mass Ratio Succinic Acid/Lactic Acid is 1.67

Keratolytic effect was assessed by observing the skin biomechanical properties modulation (skin stiffness modulation) induced by the tested agents using Atomic Force Microscopy (AFM) in skin explants.
1 explant by donor and treatment
3 distinct areas were measured by explant
A minimum of 12 measures by aera

Protocole:

Skin Explants

Skin punchs of 1.2 cm were made from breast skin of women between 35 and 43 years old. Three topical treatments of lactic acid and succinic acid alone and mixed together (ratio succinic acid/lactic acid 1.67) have been applicated on it. First and second application during 1 hour, and the third-one during 3 hours. After the treatment, the skin explants have been frozen in liquid nitrogen and stored at −80° C. Cryosections of 20 μm from treated skin explants have been made. Mechanical properties of skin explants cryosections have been assessed, at Stratum corneum and epidermis areas, by AFM measurements.

Atomic Force Microscopy (AFM)

AFM indentation experiments were carried out with a Resolve Bioscope (Bruker Nano Surface, Santa Barbara, CA) that was mounted on an inverted optical DM18 (Leica) and a top view Bruker. All measurements of skin stiffness were performed using cantilevers with conical tips. The cantilever moved with the relief of the skin surface. This deformation was quantified by the deflection of the laser beam who are correlated with biomechanical parameters of skin surface samples. Quantitative measure of elastic modulus was used to assess Stratum corneum or epidermis cell-cell junctions stiffness after treatment. Stiffness inhibition have been calculated between treated and untreated control explants.

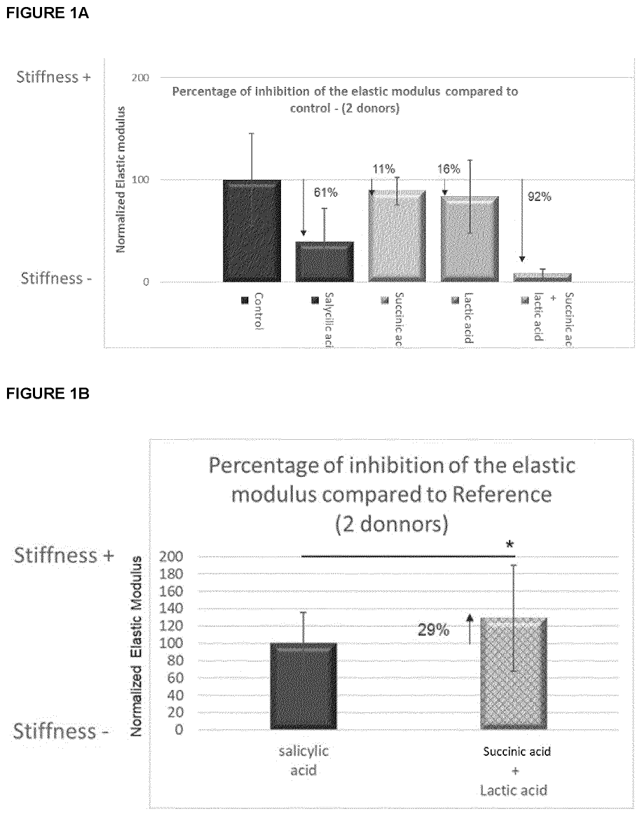

Results: Results are presented in FIGS. 1A and 1B (control—untreated-, salicylic acid 1 wt %, succinic acid 0.6 wt %, lactic acid 0.36 wt %, and lactic acid 0.36 wt %+succinic acid 0.6 wt %)

The combination of succinic acid and lactic acid induces a strong inhibition of elastic modulus of Stratum corneum (strong Stratum corneum stiffness reduction), whereas succinic acid and lactic acid alone did not show any significant effect (FIG. 1A). The inhibition is stronger than with reference keratolytic agent (salicylic acid).

The measures and analyses of cell-cell cohesion of epidermis deep layer show that the combination of succinic acid and lactic acid significantly protects epidermis deep layer cell-cell cohesion compared to reference keratolytic agent (salicylic acid) (FIG. 1B).

### Example 3: In Vitro Antibacterial Efficacy of a Composition According to the Invention

In acne skin, Cutibacterium acnes is the main trigger of acne. However, other commensals, such as Staphylococcus epidermidis, could perform an important role. It is known that dysbiosis in patients with acne is associated with a reduced number of S. epidermidis and a super-colonization by the selection of C. acnes phyllotypes in the pilosebaceous unit. This change produces different levels of activation of innate immunity, resulting in different levels of severity of inflammatory acne vulgaris. Recent researches have confirmed that the beneficial role of S. epidermidis in the physiopathology of acne occurs by limiting the super colonization of the pilosebaceous unit by C. acnes, reducing the degree of inflammation of acne vulgaris.

Time kill methodology (CEN, I.S. EN-1040; 2006: Chemical disinfectants and antiseptics quantitative suspension test for the evaluation of basic bactericidal activity of chemical disinfectants and antiseptics—Test method and requirements phase 1) was applied to quantify the microorganisms C acnes, S epidermidis and S aureus resistant to clindamycin and erythromycin after exposure to a product according to example 1 B.

Results: It was observed a reduction of more than 99.9999% (6 logs) of the initial population of C. acnes in 30 seconds, 99.9% (3 logs) of the initial microbial population of S. aureus in 5 minutes and a maximum reduction of 94.44% (1.26 log) of the initial population of S. epidermidis in 5 minutes.

The product allows an overall beneficial action for the cutaneous flora by:
reducing proven pathogens (C acnes; S aureus)
and not affecting commensals (S epidermidis).

### Examples 4-7: The Product Used in the Following In-Use Tests is a Composition According to Example 1B

### Example 4: In-Use Test to Assess Anti-Oiliness Efficacy Through Sebumeter® Measurements Immediately after Use and after 28 Days of Use

Participants: 30 healthy subjects aged between 16 and 40 years (each subject was its own control) prone to acne (acne lesions on face and trunk: subjects with at least 10 to 40 total acne lesions at face and trunk) and with oily skin (subjects with Sebumeter value >100 μg/cm² (casual level).

Protocol: The subjects had to apply the composition twice a day (morning and evening) to previously moistened skin, emulsify with water, rinse and dry for 28 days (duration of application of the investigational product).

The first application of the product (D1) was carried out by the subject in a randomized hemiface under the supervision of a trained technician. All other applications during the study were carried out at home, under normal conditions of use.

The planning of the visits was as follows:
visit 1: D1
visit 2: D8
visit 3: final visit (D29)

Subject's skin oiliness was evaluated by an instrumental measurement with Sebumeter® in the frontal region.

Results: After 28 days of product use, an oiliness control activity by the composition (−11.13%) was observed. Thus, the tested product allows controlling oiliness after 28 days of use (no increase in oiliness).

### Example 5: In-Use Test to Assess Anti-Acne Efficacy after 28 Days of Use

Participants: 30 healthy subjects aged between 16 and 40 years (each subject was its own control) prone to acne (acne lesions on face and trunk) and with oily skin.

Protocol: The subjects had to apply the composition twice a day (morning and evening) to previously moistened skin, emulsify with water, rinse and dry for 28 days.

Acne lesion count (open comedones, closed comedones, papules, pustules and nodules) was performed by a trained dermatologist on face and trunk after 28 days of use.

Results: The total acne lesions on face diminished significantly (p<0.001) after 28 days of product use (−33%), proving the anti-acne efficacy on skin face.

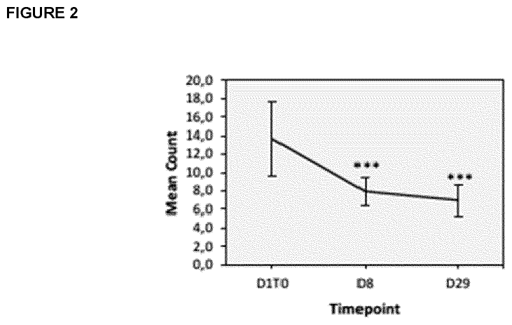

The total acne lesions on trunk diminished significantly (p<0.001) after 28 days of product use (−49%), proving the anti-acne efficacy on skin trunk (FIG. 2).

The tested product is thus non comedogenic and non acnegenic.

### Example 6: In-Use Test to Assess Pores Evolution after 7 Days and 28 Days of Use

Participants: 30 healthy subjects aged between 16 and 40 years (each subject was its own control) prone to acne (acne lesions on face and trunk) and with oily skin.

Protocol: The subjects had to apply the composition twice a day (morning and evening) to previously moistened skin, emulsify with water, rinse and dry for 28 days.

Dermatologist performed clinical evaluation of pores after 7 days and 28 days of use.

Results: The pore visibility diminished significantly (p<0.001) after 7 days of product use (−3.3%) and after 28 days of product use (−25.8%). The pore size diminished significantly (p<0.001) after 7 days of product use (−19.1%) and after 28 days of product use (−36.1%).

### Example 7: In-Use Test to Assess Subjective Evaluation of the Composition

Participants: 30 healthy subjects aged between 16 and 40 years (each subject was its own control) prone to acne (acne lesions on face and trunk) and with oily skin.

Protocol: The subjects had to apply the composition twice a day (morning and evening) to previously moistened skin, emulsify with water, rinse and dry for 28 days.

Subjects were asked to report any subjective they may have experienced on use of the test product.

Results: After 28 days of use, the composition was rated as follows:
gently cleanses the skin without drying it: 8.2/10 with 90% of subjects who rated the composition ≥5 and 76.67% ≥8;
reduces oiliness and shine: 8.37/10 with 96.67% of subjects who rated the composition ≥5 and 76.67% ≥8.

1. A topical composition comprising:
a. a combination of keratolytic agents including succinic acid and one or more alpha hydroxy acids, preferably wherein the alpha hydroxy acids consist of lactic acid;
b. one or more physiologically acceptable carriers and/or adjuvants; wherein the total amount of succinic acid and alpha hydroxy acids ranges from about 1 to about 8% by weight relative to the total weight of the composition and wherein the succinic acid/total alpha hydroxy acids mass ratio is superior or equal to 1.

2. The topical composition according to claim 1 wherein the topical composition is a topical cleansing composition.

3. The topical composition according to claim 1 wherein the carrier is water.

4. The topical composition according to claim 1 wherein the one or more alpha hydroxy acids consist of lactic acid and wherein the succinic acid/lactic acid mass ratio is superior or equal to 1.5 and inferior or equal to 4.

5. The topical composition according to claim 1 wherein the combination of keratolytic agents consists of succinic acid and lactic acid.

6. The topical composition according to claim 1 wherein the composition is in the form of a solution, lotion, foam, or gel.

7. The topical composition according to claim 1 wherein the composition comprises one or more surfactants as adjuvants.

8. The topical composition according to claim 7 wherein the composition comprises a mixture of disodium cocoyl glutamate, disodium coco glucoside citrate and decyl glucoside as surfactants.

9. The topical composition according to claim 1 wherein the composition is free from salicylic acid.

10. The topical composition according to claim 1 wherein the adjuvants comprise an anti-seborrheic agent, the anti-seborrheic agent being selected from the group consisting of 2,3-dihydroxypropyl dodecanoate, sabal extract, pumpkin seed oil, extract of urtica dioic, and mixtures thereof.

11. The topical composition according to claim 1 wherein the adjuvants comprise a pore refiner, the pore refiner being Lens esculenta seed extract.

12. Use of a topical composition as recited in claim 1 for cleansing oily skin, acne-prone skin, acne-affected skin, and/or acne-treated skin.

13. Use of a topical composition as recited in claim 1 for controlling oiliness of skin.

14. Use of a topical composition as recited in claim 1 for reducing stratum corneum stiffness while controlling epidermis integrity.

15. A cosmetic method for cleansing the skin, said method comprising:
a. applying a composition as recited in claim 1 to previously moistened skin,
b. optionally emulsifying with water,
c. rinsing the skin, and
d. drying the skin.
